(19) United States
(12) Patent Application Publication
(10) Pub. No.: US 2009/0176794 A1
(43) Pub. Date: Jul. 9, 2009

(54) 1H-INDOL-1-YL-UREA COMPOUNDS

(75) Inventors: Jean-Daniel Brion, Saint Leu La Foret (FR); Abdallah Deyine, Deville Les Rouen (FR); Alain Le Ridant, Neuilly sur Seine (FR); Catherine Harpey, Paris (FR)

Correspondence Address: THE FIRM OF HUESCHEN AND SAGE, SEVENTH FLOOR, KALAMAZOO BUILDING, 107 WEST MICHIGAN AVENUE, KALAMAZOO, MI, 49007, US

(73) Assignees: LES LABORATOIRES SERVIER, COURBEVOIE CEDEX (FR); CENTRE NATIONAL DE LA RECHERCHE SCIENTIFIQUE, PARIS CEDEX (FR); UNIVERSITE PARIS SUD, ORSAY CEDEX (FR)

(21) Appl. No.: 12/317,904

(22) Filed: Dec. 30, 2008

(30) Foreign Application Priority Data
Jan. 4, 2008 (FR) 08/00043

(51) Int. Cl. A61K 31/4439; C07D 401/02; C07D 403/02; A61K 31/454; A61K 31/506; A61K 31/501; A61P 25/28; A61P 25/16; A61P 25/22; A61P 25/24

(52) U.S. Cl. 514/252.06; 546/277.4; 546/201; 544/333; 544/238; 514/339; 514/323; 514/256

(57) ABSTRACT

Compounds of formula (I):

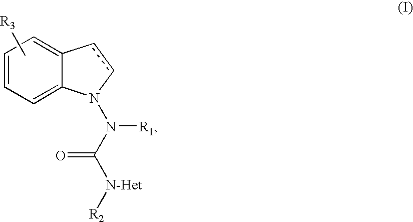

wherein:

R₁ and R₂, which may be the same or different, represent a hydrogen atom or a linear or branched (C₁-C₆)alkyl group,

R₃ represents a hydrogen or halogen atom, a linear or branched (C₁-C₆)alkyl group, or a linear or branched (C₁-C₆)alkoxy group,

Het represents a pyridyl, pyrimidinyl or piperidyl group, which are optionally substituted by one or more groups selected from halogen, linear or branched (C₁-C₆)alkyl and linear or branched (C₁-C₆)alkoxy,

—represents a single bond or a double bond,

their enantiomers and diastereoisomers, and also addition salts thereof with a pharmaceutically acceptable acid or base.

Medicinal products containing the same which are useful in the treatment of depression, anxiety, disorders of memory in the course of aging and/or neurodegenerative diseases, and in the palliative treatment of Parkinson's disease, and for adaptation to stress.

The present invention relates to new 1H-indol-1-yl-urea compounds, to a process for their preparation and to pharmaceutical compositions containing them.

The literature provides numerous examples of compounds exhibiting an eburnane structure, this being the case especially with the patent specification U.S. Pat. No. 3,454,583, which deals with vincamine (methyl(3α,14β,16α)-(14,15-dihydro-14-hydroxy-eburnamenine-14-carboxylate) and derivatives thereof with regard to their vasodilatory properties. The Patent Applications FR 2 433 528 and FR 2 381 048 present new 20,21-dinoreburnamenine compounds and the Patent Application EP 0 287 468 presents new 17-aza-20,21-dinoreburnamenine compounds. The Patent Application EP 0 658 557 describes eburnane compounds modified in the 14- and 15-positions of the eburnane skeleton. The Patent Application EP 0 563 916 describes 1H-indole-cyclohexanecarboxamide compounds.

Besides the fact that they are new, the compounds of the present invention have very valuable pharmacological properties. In particular, they have been found to be powerful selective or non-selective tyrosine hydroxylase inducers.

More specifically, the present invention relates to compounds of formula (I):

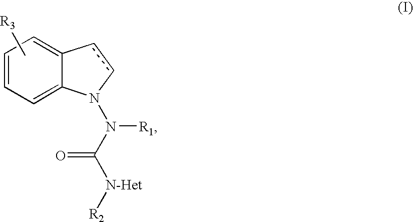

wherein:
R₁ and R₂, which may be the same or different, represent a hydrogen atom or a linear or branched (C₁-C₆)alkyl group,
R₃ represents a hydrogen or halogen atom, a linear or branched (C₁-C₆)alkyl group, or a linear or branched (C₁-C₆)alkoxy group,
Het represents a pyridyl, pyrimidinyl, piperidyl, 2-methylpyridyl, 3-methylpyridyl, 4-methylpyridyl, phenyl, benzyl, quinolyl, pyridazinyl or indolyl group, each of which may optionally be substituted by one or more groups selected from halogen, linear or branched (C₁-C₆)alkyl and linear or branched (C₁-C₆)alkoxy,
[symbol] represents a single bond or a double bond,
   it being understood that R₃ may be attached to any of the carbons of the indole/indoline nucleus that allows it,
   to their enantiomers and diastereoisomers, and also to addition salts thereof with a pharmaceutically acceptable acid or base.

Among the pharmaceutically acceptable acids there may be mentioned, without implying any limitation, hydrochloric acid, hydrobromic acid, sulphuric acid, phosphoric acid, acetic acid, trifluoroacetic acid, lactic acid, pyruvic acid, malonic acid, succinic acid, glutaric acid, fumaric acid, tartaric acid, maleic acid, citric acid, ascorbic acid, oxalic acid, methanesulphonic acid, camphoric acid etc.

Among the pharmaceutically acceptable bases there may be mentioned, without implying any limitation, sodium hydroxide, potassium hydroxide, triethylamine, tert-butylamine, lysine etc.

An advantageous embodiment relates to compounds of formula (I) wherein R₁, R₂ and R₃ each represent a hydrogen atom.

An even more advantageous embodiment of the invention relates to compounds of formula (I) wherein Het is a pyridyl, pyrimidinyl or piperidyl group.

Another particular aspect of the invention relates to compounds of formula (I) wherein Het is a pyridyl group.

Even more especially, the invention relates to the compounds of formula (I) which are:
N-(1H-indol-1-yl)-N′-(3-pyridyl)urea,
N-(2,3-dihydro-1H-indol-1-yl)-N′-(3-pyridyl)urea.

The addition salts of the preferred compounds with a pharmaceutically acceptable acid or base form an integral part of the invention.

The present invention relates also to a process for the preparation of compounds of formula (I), which process is characterised in that there is used as starting material a compound of formula (II):

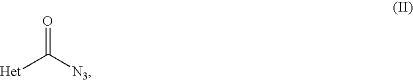

wherein Het is as defined for formula (I),
the thermal decomposition of which compound (II) causes a release of N₂, to result in the formation of an isocyanate compound of formula (III), which may be isolated:

Het-N═C═O (III).

Compound (III) is then reacted with a compound of the following formula (IV):

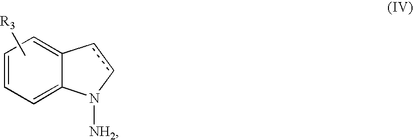

wherein R₃ is as defined for formula (I),
to yield the compounds of the invention of formula (I), which may be purified according to a conventional separation technique, which are converted, if desired, into their addition salts with a pharmaceutically acceptable acid or base and which are, where appropriate, separated into isomers according to a conventional separation technique.

The compounds of formulae (II) and (IV) are either commercially available or obtained by conventional methods of organic synthesis well known to the person skilled in the art.

The compounds of formula (I) have valuable pharmacological properties, especially that of being powerful inducers of tyrosine hydroxylase (TH). It is known that tyrosine hydroxylase is a rate-limiting enzyme which controls particularly the synthesis of neurotransmitters in central catecholaminergic and dopaminergic neurons (Zhu M.-Y. et al., Molecular Brain Research 133, (2005), 167-175). The rate of synthesis of those neurotransmitters is related especially to the appearance of tonic brain dysfunctions constituting numerous behavioural pathologies in humans, such as anxiety, psychoses, depression, stress etc. (Schloss P. et al., Pharmacology & Therapeutics 102, (2004), 47-60; Morilack D. A., et al., International Journal of Neuropsychopharmacology 7, (2004), 193-218). A deficit of noradrenaline and dopamine in the prefrontal cortex is the source of negative and cognitive symptoms especially in schizophrenia (Pira L. et al., European Journal of Pharmacology 504, (2004), 61-64).

By virtue of their ability to induce tyrosine hydroxylase, the compounds of the invention will accordingly be used therapeutically in the treatment of depression, anxiety, disorders of memory in the course of ageing and/or neurodegenerative diseases, and in the palliative treatment of Parkinson's disease, and for adaptation to stress.

The present invention relates also to pharmaceutical compositions comprising, as active ingredient, at least one compound of formula (I), an enantiomer or diastereoisomer thereof, or an addition salt thereof with a pharmaceutically acceptable acid or base, alone or in combination with one or more pharmaceutically acceptable, inert, non-toxic excipients or carriers.

The pharmaceutical compositions thereby obtained will generally be presented in a dosage form; for example, they may take the form of tablets, dragées, capsules, suppositories, or injectable or drinkable solutions and may be administered by the oral, rectal, intramuscular or parenteral route.

Among the pharmaceutical compositions according to the invention there may be mentioned more especially those that are suitable for oral, parenteral (intravenous, intramuscular or subcutaneous), per- or trans-cutaneous, intravaginal, rectal, nasal, perlingual, buccal, ocular or respiratory administration.

The pharmaceutical compositions according to the invention for parenteral injections especially include aqueous and non-aqueous sterile solutions, dispersions, suspensions or emulsions as well as sterile powders for the reconstitution of injectable solutions or dispersions.

The pharmaceutical compositions according to the invention for solid oral administration especially include tablets or dragées, sublingual tablets, sachets, capsules and granules, and for liquid oral, nasal, buccal or ocular administration especially include emulsions, solutions, suspensions, drops, syrups and aerosols.

The pharmaceutical compositions for rectal or vaginal administration are preferably suppositories or ovules, and those for per- or trans-cutaneous administration especially include powders, aerosols, creams, ointments, gels and patches.

The above-mentioned pharmaceutical compositions illustrate the invention but do not limit it in any way.

Among the inert, non-toxic, pharmaceutically acceptable excipients or carriers there may be mentioned, by way of example and without implying any limitation, diluents, solvents, preservatives, wetting agents, emulsifiers, dispersants, binders, swelling agents, disintegrants, retardants, lubricants, absorbency agents, suspension agents, colourants, flavourings etc.

The useful dosage varies according to the age and weight of the patient, the route of administration, the pharmaceutical composition used, the nature and severity of the disorder, and the administration of any associated treatments. The dosage ranges from 0.1 mg to 100 mg per day in one or more administrations.

The following Examples illustrate the invention but do not limit it in any way.

The starting materials used are known products or are prepared according to known procedures. The various Preparations yield synthesis intermediates that are useful in preparation of compounds of the invention.

The structures of the compounds described in the Examples and in the Preparations were determined in accordance with the usual spectrometric techniques (infrared, nuclear magnetic resonance, mass spectrometry etc.).

The melting points were determined using a TOTTOLI apparatus (without emergent column correction). When the compound is in the form of a salt, the melting point corresponds to that of the compound in salt form.

### PREPARATION 1

### Nicolinoyl Azide

To 2.4 ml of concentrated hydrochloric acid (37%) there are added, at 0° C., 2 g of nicotinoylhydrazide and then a solution of 2.02 g of sodium nitrite in 3.6 ml of water. The reaction mixture is stirred at 0° C. for 30 minutes and then treated with saturated sodium hydrogen carbonate solution. After extraction with diethyl ether (3 times), the organic phase is washed successively with water and with saturated sodium chloride solution before being dried over magnesium sulphate. After concentrating under reduced pressure, the expected product is obtained (G. Papeo et al., Synthesis, (2004), 2886).

Infrared(v_{cm-1}) : 2178 (v_{N3}); 1685 (v_{CO}).

### PREPARATION 2

### 2-pyridinecarbonyl azide

The compound is obtained according to a process analogous to that of Preparation 1, replacing the nicotinoylhydrazide by 2-pyridinecarbonylhydrazide.

### PREPARATION 3

### Isonicotinoyl Azide

The compound is obtained according to a process analogous to that of Preparation 1, replacing the nicotinoylhydrazide by isonicotinoylhydrazide.

### PREPARATION 4

### 1-methyl-3-piperidinecarbonyl azide

The compound is obtained according to a process analogous to that of Preparation 1, replacing the nicotinoylhydrazide by 1-methyl-3-piperidinecarbonylhydrazide.

### PREPARATION 5

### 1-methyl-2-piperidinecarbonyl azide

The compound is obtained according to a process analogous to that of Preparation 1, replacing the nicotinoylhydrazide by 1-methyl-2-piperidinecarbonylhydrazide.

### PREPARATION 6

### 1-methyl4-piperidinecarbonyl azide

The compound is obtained according to a process analogous to that of Preparation 1, replacing the nicotinoylhydrazide by 1-methyl-4-piperidinecarbonylhydrazide.

### PREPARATION 7

### 5-methoxy-2,3-dihydro-1H-indol-1-ylamine

Step A: 5-methoxy-2,3-dihydro-1H-indole

0.991 g of 5-methoxy-1H-indole is dissolved in 67 ml of glacial acetic acid at ambient temperature. 1.37 g of sodium cyanoborohydride are added in portions. After stirring at ambient temperature for 1 hour 30 minutes, the reaction mixture is dried; the residue is treated with 50 ml of water and extracted twice with dichloromethane. The organic phase is washed with saturated NaHCO₃ solution and then with saturated NaCl solution, dried over magnesium sulphate and concentrated to yield the expected product.

Step B : 5-methoxy-1-nitroso-2,3-dihydro-1H-indole

To 10 ml of 50% acetic acid solution at 0° C. there is added, all at once, 0.499 g of the compound of Step A above. At 0° C., 0.232 g of sodium nitrite dissolved in 5 ml of water is added dropwise. Once the addition is complete, the reaction mixture is stirred at 0° C. for 1 hour 30 minutes. After adding water, the aqueous phase is extracted 3 times with dichloromethane; the combined organic phases are washed with water and dried over magnesium sulphate. Evaporation to dryness yields the expected product, which is used in the subsequent reaction without purification.

Step C : 5-methoxy-2,3-dihydro-1H-indol-1-ylamine

At ambient temperature, 0.353 g of 5-methoxy-1-nitroso-2,3-dihydro-1H-indole is dissolved in 17 ml of ethanol, and 8.5 ml of water are added. 1.42 g of zinc are added, followed by 1.89 g of ammonium carbonate. After stirring vigorously for 1 hour at ambient temperature, the reduction is complete. The zinc is removed by filtration over Celite, washing with methanol. After evaporation, the residue is taken up in water and extracted 3 times with dichloromethane. The organic phase is washed with saturated NaCl solution, dried over magnesium sulphate and evaporated to dryness to yield the expected product, which is not purified for the next step.

### PREPARATION 8

### phenyl 1H-indol-1-ylcarbamate

3 g of 1H-indol-1-ylamine are dissolved in 28 ml of anhydrous dichloromethane. 2.8 g of 4-(dimethylamino)pyridine are added and the reaction mixture is cooled to 0° C. 2.85 ml of phenyl chloroformate are added dropwise and the reaction mixture is stirred at 0° C. until conversion to the carbamate is complete. After evaporating off the solvents, the residue is taken up in a pentane/dichloromethane mixture (50/50), and the insoluble material (4-(dimethylamino)pyridinium chloride) is separated off by filtration. The filtrate is concentrated to dryness and the residue obtained is dissolved in dichloromethane. The organic phase is washed 3 times with 0.1M hydrochloric acid solution and then with saturated NaCl solution. After evaporating off the solvents, the residue obtained is triturated in a pentane/diethyl ether mixture and then dried in vacuo over phosphorus pentoxide to yield the expected product.

### Melting Point : 144.5° C.

### PREPARATION 9

### phenyl quinolin-3-ylcarbamate

1.01 g of 3-aminoquinoline are dissolved in 20 ml of anhydrous dichloromethane. 1.12 g of 4-(dimethylamino)pyridine are added and the reaction mixture is cooled to 0° C. 1.14 ml of phenyl chloroformate are added dropwise and the reaction mixture is stirred at ambient temperature for 1 hour under argon. After evaporating off the solvents, 50 ml of water are added to the residue. After 15 minutes, the carbamate forms a white precipitate which, following filtration, is dried to yield the expected product.

### Melting Point : 196° C.

### PREPARATION 10

### phenyl (1H-indol-1-yl)-N-methylcarbamate

48 mg of sodium hydride are added, under inert atmosphere, to a solution of 200 mg of phenyl N-(1H-indol-1-yl) carbamate on 20 ml of tetrahydrofurane freshly distilled. After stirring for 10 minutes, 49 μl of iodomethane are added. The reaction mixture is stirred under inert atmosphere for 4 hours. After evaporation of tetrahydrofurane, 3 extractions with dichloromethane, washing with a saturated solution of NaCl, the organic phase is evaporated to dryness. The carbamate is purified by flash chromatography (ethyl acetate/cyclohexane:1/9). After evaporation of the solvents, the expected product is obtained.

### Melting Point : 112 ° C.

### EXAMPLE 1

### N-(1H-indol-1-yl)-N′-(3-pyridyl)urea

A solution of 1.14 g of the compound of Preparation 1 in 38 ml of toluene is heated at reflux under argon until the starting material has completely disappeared (2 hours). To the 3-pyridyl isocyanate obtained as intermediate and cooled to 0° C. there are added 995 mg of 1H-indolamine in 38 ml of dichloromethane. Stirring is continued at ambient temperature for 24 hours. The precipitate formed is filtered off and stirred for 12 hours in water (10 ml). After filtering and washing with pentane, the solid is taken up in a dichloromethane/methanol mixture (90/10). Following filtration, the solid is dried in vacuo over phosphorus pentoxide to yield the expected product.

### Melting Point : 202.5° C.

### EXAMPLE 2

### N-(2,3-dihydro-1H-indol-1-yl)-N′-(3-pyridyl)urea

A solution of 942 mg of the compound of Preparation 1 in 30 ml of toluene is heated at reflux under argon until the starting material has completely disappeared (1 hour 30 minutes). The 3-pyridyl isocyanate obtained as intermediate is cooled to 0° C. and 824 mg of 1-indolinamine dissolved in 30 ml of dichloromethane are added. Stirring is then continued at ambient temperature for 15 hours. The precipitate formed is filtered off and washed with diethyl ether, constituting a first batch. The mother liquors are concentrated. The residue is taken up in a minimum of dichloromethane, and diethyl ether is added. Following filtration, the precipitate formed constitutes a second batch, which is recrystallised from ethyl acetate.

### Melting Point : 197° C.

### EXAMPLE 3

### N-(5-chloro-2,3-dihydro-1H-indol-1-yl)-N′(3-pyridyl)urea

The compound is obtained according to a process analogous to that of Example 2, replacing the 1 -indolinamine by 5-chloro-1-indolinamine.

### EXAMPLE 4

### N-(5-methoxy-1H-indol-1-yl)-N′-(3-pyridyl)urea

The compound is obtained according to a process analogous to that of Example 1, replacing the 1H-indolamine by 5-methoxy-1H-indol-1-amine.

A solution of 0.281 g of the compound of Preparation 1 in 9.25 ml of toluene is heated at reflux, under argon, until conversion to the isocyanate is complete (2 hours). To this solution, cooled to 0° C., there is added 0.300 g of 5-methoxy-1H-indol-1-ylamine dissolved in 9.25 ml of dichloromethane. Stirring is continued at ambient temperature for 24 hours.

The precipitate formed is, after filtration, dissolved in dichloromethane and the organic phase obtained is washed 3 times with water. After drying over magnesium sulphate and evaporating to dryness, the residue is triturated with pentane and dried in vacuo over phosphorus pentoxide to yield the expected product.

### Melting Point : 176° C.

### EXAMPLE 5

### N-(2,3-dihydro-1H-indol-1-yl)-N′-(2-pyridyl)urea

The compound is obtained according to a process analogous to that of Example 2, replacing Preparation l by Preparation 2.

### EXAMPLE 6

### N-(1H-indol-1-yl)-N′-(4-pyridyl)urea

The compound is obtained according to a process analogous to that of Example 1, replacing Preparation 1 by Preparation 3.

A solution of 0.305 g of the compound of Preparation 3 in 8.8 ml of toluene is heated at reflux under argon until conversion to the isocyanate is complete (2 hours). To the reaction mixture, cooled to 0° C., there is added 0.265 g of 1H-indol-1-ylamine in 8.8 ml of dichloromethane. Stirring is continued at ambient temperature for 5 days. The precipitate formed is, after filtration, purified by chromatography on a silica column (ethyl acetate/cyclohexane : 60/40). After evaporating off the solvents, the residue is washed with pentane, dried in vacuo at 70° C., and then in vacuo over phosphorus pentoxide for 12 hours at ambient temperature, to yield the expected product.

### Melting Point : 138° C.

### EXAMPLE 7

### N-(1H-indol-1-yl)-N′-(1-methyl-3-piperidyl)urea

The compound is obtained according to a process analogous to that of Example 1, replacing Preparation 1 by Preparation 4.

### EXAMPLE 8

### N-(1H-indol-1-yl)-N′-(1-methyl-2-piperidyl)urea

The compound is obtained according to a process analogous to that of Example 1, replacing Preparation 1 by Preparation 5.

### EXAMPLE 9

### N-(1H-indol-1-yl)-N′-(1-methyl-4-piperidyl)urea

The compound is obtained according to a process analogous to that of Example 1, replacing Preparation 1 by Preparation 6.

### EXAMPLE 10

### N-(2,3-dihydro-1H-indol-1-yl)-N′-(1-methyl-3-piperidyl)urea

The compound is obtained according to a process analogous to that of Example 2, replacing Preparation 1 by Preparation 4.

### EXAMPLE 11

### N-(5-methyl-1H-indol-1-yl)-N′-(3-pyridyl)urea

A solution of 0.598 g of the compound of Preparation 1 in 20 ml of toluene is heated at reflux under argon until conversion to the isocyanate is complete (1 hour 30 minutes). To the reaction mixture, cooled to 0° C., there is added 0.620 mg of 5-methyl-1H-indol-1-ylamine in 5 ml of dichloromethane. Stirring is then continued at ambient temperature for 24 hours. The precipitate formed is, after filtration, washed with dichloromethane and then with pentane. It is taken up in warm ethanol; the crystals obtained are dried in vacuo over phosphorus pentoxide for 6 hours at 70° C. to yield the expected product.

### Melting Point : 197.5° C.

### EXAMPLE 12

### N-(5-methoxy-2,3-dihydro-1H-indol-1-yl)-N′-(3-pyridyl)urea

A solution of 0.270 g of the compound of Preparation 1 in 9 ml of toluene is heated at reflux under argon until conversion to the isocyanate is complete (1 hour). To the reaction mixture, cooled to 0° C., there is added 0.300 g of the compound of Preparation 7 in 5 ml of dichloromethane. Stirring is then continued at ambient temperature and, after 24 hours, conversion to the urea is complete. The precipitate formed is, after filtration, washed with dichloromethane and then with pentane. The crude product is purified by preparative plate chromatography (ethyl acetate/methanol:95/5). After filtering the eluted product over Acrodisc® GHP 0.45 μm, washing with warm ethanol yields crystals of the expected product.

### Melting Point : 177.5° C.

### EXAMPLE 13

### N-(5-chloro-1H-indol-1-yl)-N′-(4-pyridyl)urea

A solution of 0.500 g of the compound of Preparation 3 in 17 ml of toluene is heated at reflux under argon until conversion to the isocyanate is complete (1 hour 30 minutes). To the reaction mixture, cooled to 0° C., there is added 0.562 g of 5-chloro-1H-indol-1-ylamine in 17 ml of dichloromethane. Stirring is then continued at ambient temperature for 24 hours. The precipitate formed is, after filtration, purified by chromatography on a silica column (ethyl acetate/cyclohexane/methanol:60/40/5). After partially evaporating off the solvents and filtering over Acrodisc® PSF 0.45 μm, the residue is washed with pentane and dried in vacuo over phosphorus pentoxide at 70° C. for 6 hours to yield the expected product.

### Melting Point : 267.5° C.

### EXAMPLE 14

### N-(5-chloro-1H-indol-1-yl)-N′-(2-pyridyl)urea

2.45 g of carbonyldiimidazole are dissolved in 12 ml of tetrahydrofuran at 0° C. 1.023 g of 5-chloro-1H-indol-1-ylamine dissolved in 50 ml of tetrahydrofuran are added dropwise. The reaction mixture is stirred at 0° C. until conversion to the imidazolide is complete (48 hours). After removal of the solvent, the residue is dissolved in dichloromethane; the organic phase obtained is washed twice with water and once with saturated NaCl solution and is dried over magnesium sulphate. Evaporation of the solvent yields the imidazolide in the form of a viscous oil that is unstable at ambient temperature. To a solution of 0.510 g of the imidazolide in 5 ml of dichloromethane there is added dropwise, under nitrogen, 0.500 g of 2-aminopyridine. The reaction mixture is stirred at ambient temperature. After 24 hours, a precipitate is observed and stirring is continued. After 72 hours, diethyl ether is added and the solid is separated off by filtration. The solid and the filtrate are purified separately by flash chromatography and the fractions containing the expected product are combined.

Melting Point : >320° C (decomposition).

### EXAMPLE 15

### N-(1H-indol-1-yl)-N′-(2-pyrimidinyl)urea

0.408 g of the compound of Preparation 8 and 0.175 g of 2-aminopyrimidine are heated at the reflux of toluene until conversion to the urea is complete (72 hours). The precipitate obtained is separated off by filtration and then washed in a warm methanol/ethanol mixture to yield the expected product.

### Melting Point : 263° C.

### EXAMPLE 16

### N-(1H-indol-1-yl)-N′-(2-pyridyl)urea

0.500 g of the compound of Preparation 8 and 0.379 g of 2-aminopyridine are heated at 70° C. in 10 ml of anhydrous toluene for about 72 hours. The precipitate is separated off by filtration, washed with pentane and then dried to yield the expected product.

### EXAMPLE 17

### N-(1H-indol-1-yl)-N′-(phenyl)urea

At 0° C., 0.300 g of 1H-indol-1-ylamine is dissolved in 11 ml of anhydrous dichloromethane. 245 μl of commercial phenyl isocyanate dissolved in 11 ml of dichloromethane are added dropwise. The reaction mixture is allowed to come back to ambient temperature over 12 hours. After 4 days at ambient temperature, the precipitate observed is, after filtration, washed with pentane and then dried in vacuo over phosphorus pentoxide at 70° C. to yield the expected product. Melting Point 243° C.

### EXAMPLE 18

### N-(1H-indol-4-yl)-N′-(benzyl)urea

At 0° C., 0.308 g of 1H-indol-1-ylamine is dissolved in 11 ml of anhydrous dichloromethane. 300 μl of commercial benzyl isocyanate dissolved in 11 ml of dichloromethane are added dropwise. The reaction mixture is allowed to come back to ambient temperature over 12 hours. After 4 days at ambient temperature, the precipitate observed is, after filtration, washed with pentane and then dried in vacuo over phosphorus pentoxide at 70° C. to yield the expected product.

### Melting Point : 204° C.

### EXAMPLE 19

### N-(5-chloro-1H-indol-1-yl)-N′-(benzyl)urea

At 0° C., 0.301 g of 5-chloro-1H-indol-1-ylamine is dissolved in 8 ml of anhydrous dichloromethane. 245 μl of commercial benzyl isocyanate are added dropwise. The reaction mixture is allowed to come back to ambient temperature over 12 hours. After 5 days at ambient temperature, the precipitate observed is, after filtration, washed with pentane and then dried in vacuo over phosphorus pentoxide at 70° C. to yield the expected product.

### Melting Point : 218° C.

### EXAMPLE 20

### N-(5-chloro-1H-indol-1-yl)-N′-(phenyl)urea

At 0° C., 0.300 g of 5-chloro-1H-indol-1-ylamine is dissolved in 8 ml of anhydrous dichloromethane. 195 μl of commercial phenyl isocyanate are added dropwise. The reaction mixture is allowed to come back to ambient temperature over 12 hours. After 5 days at ambient temperature, the precipitate obtained is, after filtration, washed with pentane and then dried in vacuo over phosphorus pentoxide at 70° C. to yield the expected product.

### Melting Point : 247.5° C.

### EXAMPLE 21

### N-(1H-indol-1-yl)-N′-(3-pyridylmethyl)urea

2.45 g of carbonyldiimidazole are dissolved in 12 ml of tetrahydrofuran at 0° C. 1.023 g of 1H-indol-1-ylamine dissolved in 50 ml of tetrahydrofuran are added dropwise. The reaction mixture is stirred at 0° C. until conversion to the imidazolide is complete (48 hours). After evaporating off the solvent, the residue is dissolved in dichloromethane; the organic phase obtained is washed twice with water and once with saturated NaCl solution and is dried over magnesium sulphate. Removal of the solvent yields N-(1H-indol-1-yl)-1H-imidazole-1-carboxamide. To a solution of 0.600 g of N-(1H-indol-1-yl)-1H-imidazole-1-carboxamide in 2.5 ml of anhydrous dichloromethane there are added 135 μl of triethylamine. 135 μl of 3-(aminomethyl)pyridine dissolved in 2.5 ml of anhydrous dichloromethane are added dropwise; the reaction mixture is then stirred at ambient temperature for 4 days. After removal of the solvents, the residue obtained is purified by flash chromatography (ethyl acetate/cyclohexane:10/40, and then 50/50). After evaporating off the solvents, the solid obtained is washed with warm ethanol and then, after filtration, is dried to yield the expected product.

### Melting Point : 159.5° C.

### EXAMPLE 22

### N,N′-bis(5-chloro-1H-indol-1-yl)-urea

0.310 g of carbonyldiimidazole is dissolved in 5 ml of tetrahydrofuran. 0.501 g of 5-chloro-1H-indol-1-ylamine is added and the reaction mixture is stirred at ambient temperature. After 12 hours, a precipitate is observed; the reaction mixture is then heated at reflux of the tetrahydrofuran for 6 hours. After filtering off the precipitate, washing with dichloromethane and then pentane, the solid is dried in vacuo to yield the expected product.

### Melting Point : >300° C.

### EXAMPLE 23

### N,N′-bis(1H-indol-1-yl)-urea

The compound is obtained according to a process analogous to that of Example 22, replacing the 5-chloro-1H-indol-1-ylamine by 1H-indol-1-ylamine.

### Melting Point : >347° C.

### EXAMPLE 24

### N-(1H-indol-1-yl)-N′-(2-pyridylmethyl)urea

0.715 g of carbonyldiimidazole is dissolved in 5 ml of tetrahydrofuran at 0° C. 0.258 g of 1H-indol-1-ylamine dissolved in 25 ml of tetrahydrofuran is added dropwise. The reaction mixture is stirred at 0° C. for 4 hours and then left at ambient temperature. 1.2 ml of 2-(methylamino)pyridine are added to the reaction mixture; after stirring for 48 hours at ambient temperature, the solid obtained is, after filtration, washed with diethyl ether. The filtrate is concentrated until dry and purified by flash chromatography (ethyl acetate/cyclo-hexane:30/70, and then 55/45). After evaporating off the solvents, the solid obtained is washed with pentane and then, after filtration, is dried to yield the expected product.

### Melting Point : 163° C.

### EXAMPLE 25

### N-(1H-indol-1-yl)-N′-(3-quinolyl)urea

1.30 g of the compound of Preparation 9 and 0.500 g of N-amino-1H-indole are heated at the reflux of acetonitrile under argon for 20 hours. The solvent is then evaporated off in vacuo and the residue is taken up in 100 ml of aqueous sodium carbonate solution and then extracted with 3×30 ml of dichloromethane. The organic phase is dried over sodium sulphate, filtered and concentrated under reduced pressure to yield 1.50 g of crude product. Recrystallisation of the urea from ethanol allows the expected product to be obtained.

### Melting Point : 235° C.

### EXAMPLE 26

### N-(1H-indol-1-yl)-N′-(2-pyridazinyl)urea

0.409 g of phenyl 1H-indol-1-ylcarbamate and 0.152 g of 2-aminopyridazine are heated at the reflux of toluene until conversion to the urea is complete (72 hours). The precipitate obtained is, after filtration, washed with warm toluene, and then with acetone several times. The solid is finally washed with warm ethanol and then, after filtration, is dried to yield the expected product. Melting Point : 220° C.

### EXAMPLE 27

### N-(1H-indol-1-yl)-N′-(4-pyridylmethyl)urea

0.499 g of phenyl 1H-indol-1-ylcarbamate and 0.45 ml of 4-(aminomethyl)pyridine are heated at 70° C. in 10 ml of anhydrous toluene for about 36 hours. The precipitate is separated off by filtration and then washed with warm pentane. After taking up in warm ethanol and filtration, it is dried in vacuo over phosphorus pentoxide at 70° C. to yield the expected product.

### Melting Point : 182° C.

### EXAMPLE 28

### N-(1H-indol-1-yl)-N′-(6-quinolyl)urea

0.502 g of phenyl 1H-indol-1-ylcarbamate and 0.577 g of 6-aminoquinoline are heated at 70° C. in 10 ml of anhydrous toluene for about 72 hours. The precipitate is separated off by filtration, washed with pentane and then dried in vacuo over phosphorus pentoxide at 70° C. to yield the expected product.

### Melting Point : 249° C.

### EXAMPLE 29

### N-(1H-indol-1-yl)-N′-(5-quinolyl)urea

0.405 g of phenyl 1H-indol-1-ylcarbamate and 0.459 g of 5-aminoquinoline are heated at 70° C. in 10 ml of anhydrous toluene for about 36 hours. The precipitate is separated off by filtration, washed with warm acetone and then dried in vacuo over phosphorus pentoxide at 70° C. to yield the expected product.

### Melting Point : 288° C.

### EXAMPLE 30

### N-(6-chloro-3-pyridazinyl)-N′-(1H-indol-1-yl)urea

0.498 g of phenyl 1H-indol-1-ylcarbamate and 1.32 g of 3-amino-6-chloropyridazine are heated at 70° C. in 10 ml of anhydrous toluene for 10 days. The precipitate is, after filtration, dissolved in dichloromethane. The organic phase is washed 3 times with 0.2M hydrochloric acid solution and then with saturated NaCl solution. After drying over magnesium sulphate, the organic phases are concentrated to dryness and the residue obtained is taken up in diethyl ether and then dichloromethane. The urea is dried in vacuo over phosphorus pentoxide at 70° C. to yield the expected product.

Mass Spectrometry (ESI, m/z): 286 (M-1); 288 (M+1).

### EXAMPLE 31

### N-(1H-indol-1-yl)-N′-(8-quinolyl)urea

To a solution of 0.790 g of 8-aminoquinoline in 5.5 ml of toluene, cooled to 0° C., there are added, dropwise, 4.5 ml of IM trimethylaluminium solution. After stirring for 10 minutes at 0° C., the reaction mixture is allowed to return to ambient temperature and is stirred at that temperature for 1 hour. 0.390 g of phenyl 1H-indol-1-ylcarbamate is suspended in 5.5 ml of toluene at 0° C.; the aluminium complex is added to the resulting suspension using a transfer tube. After returning to ambient temperature, the reaction mixture is heated at 65° C. until conversion to the urea is complete (1 hour 30 minutes). The reaction mixture is hydrolysed with water, and the toluene is removed by evaporation in vacuo. The residue is taken up in water and extracted with dichloromethane. The organic phase is washed 3 times with 0.5M hydrochloric acid solution and then with saturated NaCl solution and is then dried over magnesium sulphate. After removal of the solvents, the residue is recrystallised from a pentane/diethyl ether mixture (90/10). After filtration, the crystals obtained are dried in vacuo over phosphorus pentoxide at 70° C. to yield the expected product.

### Melting Point : 201° C.

### EXEMPLE 32

### N-(1H-indol-1-yl)-1-methyl-N′-(3-pyridyl)urea

To a solution of 100 mg of phenyl N-(1H-indol-1-yl)-N-methylcarbamate in 20 ml of anhydride tetrahydrofurane, are added 54 mg of 3-aminopyridine and 220 mg of (4-dimethylamino)pyridine. The solution is stirred for 3 days under reflux of the dimethylformamide. After 3 extractions with ethyl acetate, washing with a saturated solution of NaCl, the organic phase is evaporated to dryness. The urea is purified par flash chromatography (ethyl acetate/cyclohexane :3/7) to yield the expected product.

Spectrométrie de masse (ESI, m/z: 289.1 (M+23).

### EXEMPLE 33

### N-(1H-Indol-1-yl)-3-methyl-3-(3-pyridyl)urea

To a solution of 313 mg of phenyl (1H-indol-1-yl)carbamate in 10 ml of anhydride toluene, are added 135 g of 3-(méthylamino)pyridine. The solution is stirred under argon during 2 days at the reflux of toluene then leave to room temperature for 2 days. The obtained precipitate is filtered to yield the expected product.

Point de fusion: 168 ° C.

### EXEMPLE 34

### N-(1H-Indol-1-yl)-1,3-dimethyl-3-(3-pyridyl)urea

80 mg of sodium hydride are added, under inert atmosphere, to a solution of 252 mg of 1-(1H-indol-1-yl)-3-(pyridin-3-yl)urea in 20 ml of tetrahydrofurane freshly distilled. After agitation for 10 minutes, 124 μl of iodomethane are added. The reaction mixture is stirred under inert atmosphere for 2 days. After evaporation of tetrahydrofurane, 3 extractions with dichloromethane, washing with a saturated solution of NaCl, the organic phase is evaporated to dryness. The urea is purified by flash chromatography (dichloromethane/methanol:95/5) to yield the expected product.

Point de fusion: 99 ° C.

### PHARMACOLOGICAL STUDY OF COMPOUNDS OF THE INVENTION

### EXAMPLE A

### Induction of Tyrosine Hydroxylase

A search is made among the compounds for those which are capable of bringing about an increase in the tyrosine hydroxylase (TH) protein in the locus coeruleus (LC) of the brain of the Balb/C mouse.

The animals used are male mice of the pure Balb/C strain (Charles River Laboratories) aged from 7 to 8 weeks at the time of treatment.

The mice are given a single injection, by the intraperitoneal route, of the compound under test, dissolved in 0.04M HCl solution (corresponding control: 0.004M HCl), if the compound is sufficiently soluble, or in olive oil 90%/DMSO 10% (corresponding control: olive oil 90%/DMSO 10%) for compounds that are insoluble in an aqueous medium.

Three days after the injection of each compound, all the animals are sacrificed by decapitation. The brains are removed and then frozen in liquid nitrogen and stored at −80° C.

Coronal sections 8 microns thick are taken along the posterior-anterior axis of the LC and fixed. The sections are transferred onto Immobilon-P membranes. The TH is measured by immunodetection and image analysis.

### Results:

The results for TH induction in the LC are given in Table I below.

TABLE I
Measurement of the amount of TH in the various LC sections,
numbered from 1 to 8 in the anterior-to-posterior direction,
after i.p. administration (20 mg/kg)
The results are expressed in %, relative to the mean
value of the control group¹

| % | 1 | 2 | 3 | 4 | 5 | 6 | 7 | 8 |
|---|---|---|---|---|---|---|---|---|
| Example 1 | 66 | 85 | 74 | 64 | 45 | 40 | 8 | 2 |
| Example 2 | 63 | 77 | 64 | 57 | 34 | 15 | 8 | 2 |
| Example 6 | 65 | 81 | 59 | 33 | 20 | 13 | 8 | 2 |
| Example 24 | 74 | 78 | 68 | 45 | 29 | 19 | 5 | 7 |

¹animals treated with the same carrier

### EXAMPLE B

### Predicted Metabolic Stability

The predicted metabolic stability is tested by incubation of the compounds at a concentration of 10⁻⁷M in the presence of mouse, rat or human hepatic microsomes (0.33 mg of prot/ml). After addition of NADPH (nicotinamide adenine dinucleotide phosphate, reduced form), samples are taken at 0, 5, 15, 30 and 60 minutes. The enzymatic reaction is stopped using methanol (V/V). The protein is precipitated by centrifugation and the supernatant is analysed by LC-MS-MS.

The good metabolic stability of the compounds makes it possible to envisage treatment per os.

TABLE II
% metabolic stability predicted using hepatic microsomes

| | Mouse | Rat | Human |
|---|---|---|---|
| Example 1 | 78 | 78 | 72 |
| Example 2 | 85 | 55 | 98 |
| Example 6 | 77 | 73 | 77 |
| Example 13 | 84 | 89 | 82 |
| Example 24 | 72 | 69 | 67 |

Compounds having good predicted metabolic stability in humans have the advantage that they can be administered via the oral route (between 67 and 82%).

### EXAMPLE C

### Predicted Crossing of the Blood-Brain Barrier (BBB)

The substances are incubated at 20 μM in the upper compartment of a double container, the upper compartment being separated from the lower compartment either solely by a polycarbonate filter or by the same filter covered with confluent endothelial cells from bovine capillaries. Evaluation of the permeability kinetics is carried out by means of LC-MS-MS quantification of the unchanged substance in the lower compartment after 10, 20, 30, 40 and 60 minutes.

The compounds tested exhibit a generally high degree of crossing of the BBB, which promotes access to the neurological target. The results are given in the form of categories: high, intermediate, low. Accordingly, Example 1 exhibits a high degree of crossing of the BBB.

### EXAMPLE D

### Increased Phosphorylation of ERK in Specific Brain Structures Responsible for the Activity

ERK is a kinase which, in its phosphorylated p-ERK form, activates certain proteins, including tyrosine hydroxylase. The latter, when it has been both increased, and activated by phosphorylation, promotes the synthesis of dopamine and noradrenaline, a lack of which lies at the origin of neurological and psychiatric pathologies such as Parkinson's disease, the deficit forms of schizophrenia, depression etc.

It is known that administration of an ERK phosphorylation inhibitor produces depressive behaviour in the mouse (Duman C. H. et al., Biological Psychiatry 61, (2007), 661-670). Exposure of rats to stress is accompanied by a reduction in ERK phosphorylation (Yang C.-H. et al., The Journal of Neuroscience 24(49), (2004), 11029-11034).
ERK is a critical player in the synaptic and neuronal plasticity that is necessary for learning and good memory function. Inhibition of its phosphorylation brings about disturbances of cognition (Adams J. P. et al., Annual Review of Pharmacology and Toxicology 42, (2002), 135-163).
Administration of phencyclidine, which reproduces the symptoms of schizophrenia in humans and in animals, inhibits ERK phosphorylation both in vitro and in vivo (Enomoto T. et al., Molecular pharmacology 68, (2005), 1765-1774).

The role of the compound of Example 1 in ERK phosphorylation has been demonstrated in vitro and in ex vivo structures:

In vitro
1. Method

Various forms of non-phosphorylated recombinant ERK (ERK1 and ERK2) and of phosphorylated recombinant ERK (p-ERK1 and p-ERK2) are fixed separately on “chips” and studied in the Biacore® system. The direct interaction of the compound of Example 1 at different concentrations is studied.
2. Results

The compound of Example 1 binds strongly to ERK1 and ERK2 in dose-dependent manner. This binding is inhibited if the corresponding phosphorylated ERK is added to the medium.

### Ex Vivo in Pig Brain Tissues

b 1. Method

The various brain structures are isolated, treated and suspended in a medium without phosphate, after inactivation of the phosphatases of the tissues. The tissues are cleared of their own ERK and p-ERK and placed in the presence of specific recombinant forms of ERK1, ERK2, p-ERK1 and p-ERK2.

The phosphorylation capacities are studied in the presence and absence of the compound of Example 1 by means of antibodies that allow evaluation of non-phosphorylated and phosphorylated ERK1 and ERK2.
2. Results

The compound of Example 1, at a concentration of 3×10⁻⁹M, increases the phosphorylation of ERK1 and ERK2 preferentially in the hippocampus and the prefrontal cortex, and also that of ERK2 in the striatum.

### EXAMPLE E

### Pharmaceutical Composition

### Formula for the Preparation of 1000 Tablets Each Containing 10 mg of Active Ingredient

Compound of Example 1 . . . 10 g
Hydroxypropylcellulose . . . 2 g
Wheat starch . . . 10 g
Lactose . . . 100 g
Magnesium stearate . . . 3 g
Talc . . . 3 g

1. A compound selected from those of formula (I): 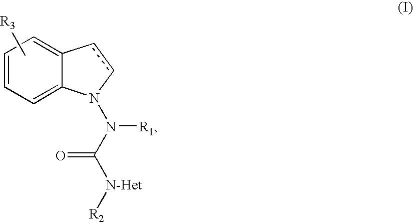
wherein
R₁ and R₂, which may be the same or different, represent a hydrogen atom or a linear or branched (C₁-C₆)alkyl group,
R₃ represents a hydrogen or halogen atom, a linear or branched (C₁-C₆)alkyl group, or a linear or branched (C₁-C₆)alkoxy group,
Het represents a pyridyl, pyrimidinyl, piperidyl, 2-methylpyridyl, 3-methylpyridyl, 4-methylpyridyl, phenyl, benzyl, quinolyl, pyridazinyl or indolyl group, each of which may optionally be substituted by one or more groups selected from halogen, linear or branched (C₁-C₆)alkyl and linear or branched (C₁-C₆)alkoxy, [symbol]
represents a single bond or a double bond,
it being understood that R₃ may be attached to any of the carbons of the indole/indoline nucleus that allows it,
its enantiomers and diastereoisomers, and addition salts thereof with a pharmaceutically acceptable acid or base.

2. The compound of claim 1, wherein R₁, R₂ and R₃ each represent a hydrogen atom.

3. The compound of claim 1, wherein Het is a pyridyl, pyrimidinyl or piperidyl group.

4. The compound of claim 1, wherein Het is a pyridyl group.

5. The compound of claim 1 which is selected from N-(1H-indol-1-yl)-N′-(3-pyridyl)urea and N-(2,3-dihydro-1H-indol-1-yl)-N′-(3 -pyridyl)urea.

6. A pharmaceutical composition comprising as active ingredient at least one compound of claim 1 in combination with one or more inert, non-toxic, pharmaceutically acceptable excipients or carriers.

7. A method of treating a living animal body, including a human, afflicted with a condition selected from depression, anxiety, disorders of memory associated with aging and/or neurodegenerative diseases, Parkinson's disease, and stress, comprising the step of administering to the living animal body, including a human, a therapeutically effective amount of a compound of claim 1.
